(19) United States
(12) Patent Application Publication
(10) Pub. No.: US 2020/0102562 A1
(43) Pub. Date: Apr. 2, 2020

(54) Beta-TUBULIN, Beta-TUBULIN GENE AND APPLICATION OF GENE SEGMENT THEREOF

(71) Applicant: Nanjing Agricultural University, Nanjing City (CN)

(72) Inventors: Mingguo ZHOU, Nanjing City (CN); Xiushi SONG, Nanjing City (CN); Kaixin GU, Nanjing City (CN); Yiping HOU, Nanjing City (CN); Yabing DUAN, Nanjing City (CN); Jianxin WANG, Nanjing City (CN)

(21) Appl. No.: 16/576,863

(22) Filed: Sep. 20, 2019

(30) Foreign Application Priority Data
Sep. 21, 2018 (CN) 201811105553.8

(51) Int. Cl. C12N 15/113; C07K 14/37; C12N 15/80; C12N 15/82

(52) U.S. Cl. CPC C12N 15/113; C07K 14/37; C12N 2310/141; C12N 15/8201; C12N 2310/11; C12N 15/80

(57) ABSTRACT

The invention provides a β-tubulin segment of a β-tubulin gene, and also provides an application of the segment in controlling plant diseases and/or enhancing plant disease resistance, inhibiting pathogenic fungal development and pathogenicity, and improving the drug sensitivity of pathogens to tubulin binding agents. The invention also provides an in-vitro interference preparation including βTubdsRNA segments of the above-mentioned β-tubulin gene, and an application of the in-vitro interference preparation in breeding a transgenic plant resistant variety. The RNA interference technology of the β-tubulin gene of the present invention has green and safe advantages of increasing the drug sensitivity of pathogenic fungi, reducing the level of drug resistance, interfering with pathogenicity, enhancing plant disease resistance, controlling a variety of plant diseases, improving the drug sensitivity to a chemical agent (carbendazim), and prolonging the dsRNAs drug retention period by the carbendazim.

### CROSS REFERENCE TO RELATED APPLICATIONS AND DOCUMENTS

This application claims priority to Chinese application number 201811105553.8, filed Sep. 21, 2018, entitled “β-TUBULIN, β-TUBULIN GENE AND APPLICATION OF GENE SEGMENT THEREOF”, which is incorporated herein by reference in its entirety.

Further, this patent application incorporates by reference the Sequence Listing file enclosed herewith having the file name “867-9_Seq Listing_ST25.txt” which is comprised of 16,062 bytes and has a date of creation of Nov. 6, 2019.

### TECHNICAL FIELD

The present invention belongs to the field of genetic engineering technology, and relates to a Fusarium β-tubulin gene, RNAi vectors of the gene, in-vitro preparation of the gene βTubdsRNA and an application of the gene in controlling plant diseases.

### BACKGROUND

Carbendazim, also called benzimidazole-2-carbamate, belonging to benzimidazole fungicides, is a tubulin binding agent, and it is featured by high efficiency and low toxicity, and has the effects of strong systematic property as well as protecting and treating crops. Since the 1970s, carbendazim has become one of the efficient fungicides in controlling Fusarium head blight, rice blast, gray mold and other major diseases of crops. However, the long-term use of benzimidazole fungicides leads to challenging problems, for example, fungicide resistance. The harm caused by chemicals and the length of time that they will remain in the environment is of great concern to the future and safety of human. Therefore, it is necessary to develop safer and more environmentally friendly fungal control methods, as well as to assure their widespread use.

Carbendazim acts as mitotic inhibitors by selectively binding to the β-tubulin monomer and consequently disrupting the microtubule polymerization process (Howard&Aist, 1980). Unlike most filamentous fungi, such as Magnaporthe oryzae and Botrytis cinema, which had only one β-tubulin gene, there were two copies of β-tubulin in Fusarium genome, namely, β1-tubulin and β2-tubulin genes. The β2-tubulin plays an essential role in growth, development and pathogenicity of fungus. Accumulating evidence proved that point mutations in β2-tubulin have led to Fusarium resistance to benzimidazoles. In most case, mutations at codons 167, 198 and 200 in the β2-tubulin gene have been given rise to benzimidazole-resistance. The expression level of β2-tubulin is positively correlated with the level of fungicide resistance; the expression quantity and biological function of β1-tubulin are mainly achieved by controlling β2-tubulin gene. Therefore, to reduce the expression of β2-tubulin may reduce the expression quantity of β-tubulin, increase the sensitivity of Fusarium to benzimidazole and to lower the level of fungicide resistance; meanwhile, β-tubulin plays an important role in the growth and development of Fusarium, therefore, to reduce the expression of β-tubulin gene may lower the fitness of Fusarium.

In recent 10 years, RNA interference has become a scientific research hotspot, and the technique has achieved a great number of outcomes in controlling plant diseases, and may effectively inhibit the occurrence of diseases by specifically interfering with key genes of pathogens in a way of host-induced gene silencing (HIGS) or spray-induced gene silencing (SIGS). HIGS is a process that genes of pathogen are built into a cervical-loop structure and transformed into plants for transcription, the RNA interference mechanism in plants produces siRNA of target genes of pathogen, when pathogen invade transgenic plants to utilize plant nutrients, siRNA caused by plants is wrapped by Golgi apparatus to form multivesicular bodies, afterwards, plant cells are secreted via exosomes, multivesicular bodies enter into pathogen cells by fungal endocytosis, enabling the RNAi mechanism of pathogen to silence its target genes (Koch&Kogel, 2014; Cai et al., 2018). In-vitro spray of long RNA-induced gene silencing has been verified in Botrytis cinerea and Fusarium graminearum (Wang, etc., Bidirectional cross-kingdom RNAi and fungal uptake of external RNAs confer plant protection. NatPlants. 2016 (2)16151; Koach, etc., An RNAi-Based Control of Fusarium asiaticum Infections Through Spraying of Long dsRNAs Involves a Plant Passage and Is Controlled by the Fungal Silencing Machinery. Plos Pathogens. 2016(12)e1005901.).

RNA-interfered target gene is a key to determining the level of RNA interference effect. Tubulin encoded by β-tubulin gene is a kind of structural protein to inhibit the formation of spindle body and interfere with mitosis and material transportation, is an important component of cytoskeleton, and tubulin participates in cell division, intracellular material transportation, cell differentiation and development and other biological process. β-tubulin has high conservation in different fungi, an RNAi vector is built specific to Fusarium asiaticum β-tubulin gene sequence to screen an effective RNAi segment reducing pathogenicity of pathogen, thus decreasing the occurrence of diseases; Fusarium asiaticum, Fusarium graminearum, Fusarium oxysporum, Fusarfum tricinctum, Fusarium fujikuroi, Botrytis cinerea, Magnaporthe oryzae and Colletotrichum higginsianum are directly or indirectly induced to produce a RNA interference mechanism of β-tubulin by transgenic technology or spraying βTubdsRNA in vitro, thus influencing the normal growth, development and pathogenicity of pathogen, which provides a key technique for the reduction of pesticide application and development of nucleic acid pesticides for controlling plant diseases. Therefore, the researched RNA interference technology of the β-tubulin gene of the present invention has green and safe advantages of increasing the drug sensitivity of pathogenic fungi, reducing the level of fungicide resistance, interfering with pathogenicity, enhancing plant disease resistance, controlling Fusarium head blight, rice blast, gray mold, anthracnose and the specificity of other plant diseases.

### SUMMARY

An objective of the present invention is to provide an effective RNA interference segment of a β-tubulin gene of pathogens for enhancing fungal drug sensitivity to β-tubulin binding agent or reducing drug resistance and improving plant disease resistance, thus being applied in plant resistance and in vitro interference; another objective is to develop a βTubdsRNA nucleic acid pesticide, used for controlling plant diseases caused by fungi and enhancing fungal drug sensitivity to the β-tubulin binding agent. The β-tubulin gene and interference segment thereof in the present invention may serve as a target for HIGS and SIGS to be introduced into plants (including wheat, paddy, grape, etc.) by means of transgenosis, plant viruses and other genetic engineering techniques, or to be sprayed onto the surface of host plants by in-vitro interference, so as to silence the expression of β-tubulin gene of Fusarium, thus reducing the Fusarium resistance to carbendazim and controlling the occurrence of diseases, and a novel disease control technique is provided.

To achieve the above objectives, the thought of the present invention is to build an RNAi vector specific to β-tubulin gene sequence and screen an effective RNAi segment for reducing virulence of pathogen, thus decreasing the occurrence of diseases; based upon the theory of gene interference, βTubdsRNA is sprayed or a βTubRNAi vector is built by means of transgenic technology and transformed into plants to directly or (and) indirectly induce Fusarium, Botrytis cinerea, Magnaporthe oryzae and Colletotrichum higginsianum to induce RNA interference of β-tubulin gene, thus bringing abnormal growth and development of pathogen and improving the sensitivity to benzimidazole agents, which provides a key technique for the reduction of pesticide application and development of nucleic acid pesticides for controlling plant diseases.

Therefore, the present invention provides a β-tubulin segment of a β-tubulin gene, and the segment is 15-30 nt siRNA obtained by performing RNase random digestion on the βTubdsRNA synthesized with a combination of segments βTubdsRNA and dsRNA or full-length or partial length of DNA chain as a template.

In the present invention, the β-tubulin gene is from Fusarium asiaticum, Fusarium graminearum, Fusarium oxysporum, Fusarium fujikuroi, Fusarfum tricinctum, Botrytis cinerea, Magnaporthe oryzae or Colletotrichum higginsianum.

The βTubdsRNA segments are 4 different segments obtained by dividing cDNA of the β-tubulin gene, respectively named βTub-1, βTub-2, βTub-3 and βTub-4. When β-tubulin gene is from Fusarium asiaticum (as shown in SEQ ID NO. 2 full length: 1686 nt) or Fusarium graminearum (as shown in SEQ ID NO.3, full length: 1686 nt), βTub-1 (cDNA start-stop loci: 1 nt-482 nt), βTub-2 (cDNA start-stop loci: 460 nt-984 nt), βTub-3 (cDNA start-stop loci: 917 nt-1405 nt) and βTub-4 (cDNA start-stop loci: 1345 nt-1686 nt). Particularly preferably, βTub-3 at cDNA start-stop loci: 917 nt-1405 nt of β-tubulin gene has particularly outstanding effect.

When β-tubulin gene is from Magnaporthe oryzae (as shown in SEQ ID NO.4, full length: 1779 nt), βTub-1 (cDNA start-stop loci: 1 nt-482 nt), βTub-2 (cDNA start-stop loci: 460 nt-984 nt), βTub-3 (cDNA start-stop loci: 922 nt-1409 nt) and βTub-4 (cDNA start-stop loci: 1345 nt-final base).

When β-tubulin gene is from Botrytis cinerea (as shown in SEQ ID NO.5, full length: 1281 nt), βTub-1 (cDNA start-stop loci: 1 nt-482 nt), βTub-2 (cDNA start-stop loci: 460 nt-984 nt), βTub-3 (cDNA: 727 nt-1209 nt), βTub-4 (cDNA start-stop loci: 1150 nt-final base).

When β-tubulin gene is from Colletotrichum higginsianum (as shown in SEQ ID NO.6, full length: 1344 nt), βTub-1 (cDNA start-stop loci: 1 nt-482 nt), βTub-2 (cDNA start-stop loci: 460 nt-984 nt), βTub-3 (cDNA start-stop loci: 755 nt-1242 nt), βTub-4 (cDNA start-stop loci: 1150 nt-final base).

According to a preferred embodiment, the dsRNA segment is a β-tubulin gene dsRNA segment synthesized with full-length βTubcDNA as a template, the sequence behind T (thymine) is substituted by a sequence of the dsRNA segment and its corresponding template U (uracil).

β-tubulin gene from Fusarium is set as an example, and a construction method of the dsRNA segment is as follows:

(1) cDNA ofβ-tubulin gene is divided into 4 different segments, respectively named βTub-1 (cDNA start-stop loci: 1 nt-482 nt), βTub-2 (cDNA start-stop loci: 460 nt-984 nt), βTub-3(cDNA start-stop loci: 917 nt-1405 nt) and βTub-4 (cDNA start-stop loci: 1345 nt-1686 nt), a specific PCR primer is designed to amplify a forward sequence of each segment, and each primer sequence is as follows:

2Tub-1F:
(SEQ ID NO. 7)
GGGGACAAGTTTGTACAAAAAAGCAGGCTCTCCTTCATTTTACTTTTAG
GC
2Tub-1R:
(SEQ ID NO. 8)
GGGGACCACTTTGTACAAGAAAGCTGGGTAGCTCGGCACCCTCGGTGTA
AT
2Tub-2F:
(SEQ ID NO. 9)
GGGGACAAGTTTGTACAAAAAAGCAGGCTAACTGGGCCAAGGGTCATTA
CAC
2Tub-2R:
(SEQ ID NO. 10)
GGGGACCACTTTGTACAAGAAAGCTGGGTGACCAGTCAGAGGGGCAAAT
CC
2Tub-3F:
(SEQ ID NO. 11)
GGGGACAAGTTTGTACAAAAAAGCAGGCTAGCTCGCTGTTAACATGATT
CCG
2Tub-3R:
(SEQ ID NO. 12)
GGGGACCACTTTGTACAAGAAAGCTGGGTTGCAAGTTGGACTGGGCCTC
GG
2Tub-4F:
(SEQ ID NO. 13)
GGGGACAAGTTTGTACAAAAAAGCAGGCTGGCTTTCTTGCATTGGTACA
CAAG
2Tub-4R:
(SEQ ID NO. 14)
GGGGACCACTTTGTACAAGAAAGCTGGGTTTAGAAATCGTGCTTAAAAA
CAC

50 μL PCR reaction system: water is added to 1 μL of a first-strand cDNA template of β-tubulin gene, 5 μL of 10×LA PCR buffer, 4 μL of 10 mM dNTP, 1 μL of forward and reverse primers respectively, 0.5 μL of LATaq enzyme to 50 μL (reagents for PCR reaction are purchased from TaKaRa (Da Lian)). PCR reaction conditions are: initial denaturation for 3 min at 95° C.; 95° C. 20 s, 56° C. 30 s, 72° C. 30 s for three cycles; extension for 10 min at 72° C.

PCR products are respectively mixed with a commercial donor vector pDONR201 after extracted by gel to form an entry vector containing a new site attL1/attL2, then the entry vector is mixed with a target vector pDestination respectively for specific recombination, and reaction products are transformed into Escherichia coli competence to form an interference expression vector of pβTubRNAi series: pβTubRNAi-1, pβTubRNAi-2, pβTubRNAi-3 and pβ-4. The interference vector respectively contains βTub-1, βTub-2, βTub-3 and βTub-4 of the cDNA segment, the sequence of the dsRNA segment is the same as that of the above cDNA segment whose sequence behind T (thymine) is substituted by U (uracil).

As a preferred embodiment, the combination of the dsRNA segment is a gene segment obtained by an RNAi Kit with the combination of the foregoing multiple dsRNA segments as a template of the synthesized segment.

The present invention further provides an application of the β-tubulin gene segment in controlling plant diseases and/or enhancing plant disease resistances.

And, an application of the β-tubulin gene segment in inhibiting the development and pathogenicity of pathogens is provided.

And, an application of the β-tubulin gene segment in enhancing drug sensitivity of pathogens to tubulin binding agents is provided.

Further, the present invention also provides an in-vitro interference preparation, and the in-vitro interference preparation includes βTubdsRNA segments of the above β-tubulin gene.

As an optional embodiment, the preparation is prepared as follows:

### (1) Preparation of a βTubdsRNA Preparation

1) Full-length β-tubulin cDNA serves as a template to synthesize 2 kinds of 850 bp dsRNAIβTub-5 (1 nt-850 nt) and 850 bp βTub-6(836 nt-1686 nt) of the β-tubulin gene, the dsRNA synthesis is conducted by a MEGAscript®RNAi Kit AM1626 kit from Invitrogen, sequences of the 2βTubdsRNA are the same as that of the corresponding template whose sequence behind T (thymine) is substituted by U (uracil);

A specific PCR primer is designed to amplify a forward sequence of each segment, and the primer sequence is as follows:

2Tub-5F:
(SEQ ID NO. 15)
TAATACGACTCACTATAGGGAGACTCCTTCATTTTACTTTTAGGC
2Tub-5R:
(SEQ ID NO. 16)
TAATACGACTCACTATAGGGAGAAAATCAGGTAGTTGAGATCGGC
2Tub-6F:
(SEQ ID NO. 17)
TAATACGACTCACTATAGGGAGACCACCGTCATGGCCGGTGTGAC
2Tub-6R:
(SEQ ID NO. 18)
TAATACGACTCACTATAGGGAGATTTAGAAATCGTGCTTAAAAAC

50 μL PCR reaction system: water is added to 1 μL of a first-strand cDNA template of β-tubulin gene, 5 μL of 10×LA PCR buffer, 4 μL of 10 mM dNTP, 1 μL of forward and reverse primers respectively, 0.5 μL of LATaq enzyme to 50 μL (reagents for PCR reaction are purchased from TaKaRa (Da Lian)). PCR reaction conditions are: initial denaturation for 3 min at 95° C.; 95° C. 20 s, 56° C. 30 s, 72° C. 30 s for three cycles; extension for 10 min at 72° C.

After PCR products are extracted by gel, βTub-5(1nt-850 nt) and βTub-6(836 nt-1686 nt) respectively serve as a template to synthesize the corresponding dsRNA (specific steps are as shown in the specification of the kit) via a MEGAscript®RNAi Kit AM1626 kit from Invitrogen to obtain βTubdsRNA, the sequence of each βTubdsRNA is the same as that of the corresponding template whose sequence behind T (thymine) is substituted by U (uracil);

2) Any of βTub-1, βTub-2, βTub-3 and βTub-4 or any combination of different segments serves as a template of the synthesized segment of the in-vitro interference dsRNA to synthesize 14 kinds of βTubdsRNA, and the dsRNA synthesis is performed by a MEGAscript®RNAi Kit AM1626 kit from Invitrogen, the sequence of each of the 14 βTubdsRNA is the same as that of the corresponding template whose sequence behind T (thymine) is substituted by U (uracil);

3) Full-length β-tubulin cDNA serves as a template to synthesize full-length βTubdsRNA by aMEGAscript®RNAi Kit 1626 from Invitrogen, and ShortCut®III (from NEB, and steps are as shown in the specification of the kit) is used for digestion to produce 15-30 nt siRNA randomly.

The above 16 kinds βTubdsRNA and a 15-30 nt siRNA are respectively diluted by an ammonium acetate buffer solution, and preserved at −20° C. for further use.

The present invention further provides an application of the above in-vitro interference preparation in breeding transgenic plant resistant variety.

Specific to different types of fungal diseases, such as wheat (barley) gibberellic disease, Fusarium fujikuroi, Fusarium oxysporum, Fusarfum tricinctum, cucumber (grape) Botrytis cinerea, Magnaporthe oryzae, Colletotrichum higginsianum, in the present invention, β-tubulin of these fungi or homologous gene thereof serves as a target gene to be applied in improving disease resistance of wheat, barley, paddy and other crops as well as in decreasing pathogenicity and drug resistance of Fusarium graminearum, Fusarium oxysporum, Fusarfum tricinctum, Fusarium fujikuroi, Botrytis cinerea, Magnaporthe oryzae, Colletotrichum higginsianum and other pathogens. Through experiments, the present invention proves that the RNA interference technology of β-tubulin gene has green and safe advantages of increasing the drug sensitivity of pathogenic fungi, reducing the level of drug resistance, interfering with pathogenicity, enhancing plant disease resistance, controlling the specificity of multiple plant diseases.

### BRIEF DESCRIPTION OF THE DRAWINGS

FIG. 1 is a schematic diagram showing that a β-tubulin gene is divided into 4 different segments and pβRNAi vectors of corresponding fungi are built,

FIG. 2 shows phenotypes of a carbendazim tolerant strain NJ003 and different βTubRNAi strains after being cultured in PDA media for 3 d,

FIG. 3 shows Southern bolt detection of βTubRNAi strains,

In the figure, NJ003 is fungus of a negative control, and the one with one strip is a single-copy gene integration transformant,

FIG. 4 is an analysis on pathogenicity of βTubRNAi strains inoculated on Yumai 35 wheat in seedling stage,

In the figure, * denotes significant difference (P<0.05) between an experimental group and a control group, the control group denotes the strain NJ003,

FIG. 5 shows measurement on field efficacy of a high-resistant strain NJ003 and a βTubRNAi transformant after being sprayed 50% wettable powder,

In the figure: different letters denote significant difference among samples (P<0.05),

FIG. 6 shows measurement on field efficacy of βTubdsRNA to Fusarium. In the figure, the concentration of βTubdsRNA and siRNA is 30 ng μL-1, the concentration of carbendazim is 1 ng μL-1, and the concentration of a carbendazim preparation is 150 ng μL-1,

FIG. 7 shows an analysis on the concentration and anti-fungal efficiency of RNA,

FIG. 8 shows measurement on in-vitro interference of βTubdsRNA to multiple pathogens,

FIG. 9 shows measurement on βTubdsRNA activity against Fusarium, Magnaporthe oryzae, Botrytis cinerea and Colletotrichum higginsianum on living plants,

In the figure: different letters denote significant difference among samples (P<0.05),

FIG. 10 shows a homologous analysis on β-tubulin gene in different fungi, and

FIG. 11 is a schematic diagram showing transformation of vectors in Arabidopsis thaliana.

### DETAILED DESCRIPTION

Embodiments below are intended to non-restrictively explain the technical solution of the present invention.

In the present invention, unless otherwise specified, “%” denoting concentration is weight percentage, “parts” are parts by weight.

The present invention relates to the following media, and ingredients thereof are respectively as follows:

PDA medium: 200 g potato was boiled for 15 min to take lixivium, then distilled water was added to 20 g glucose and 15 g agar to 1000 ml for sterilization for 20 min at 121° C.;

SNA medium: distilled water was added to 0.1% KH2PO4, 0.1% KNO3, 0.05% MgSO4.7H 2O, 0.05% KCl, 0.02% glucose and 0.02% sucrose to 1000 ml.

### Embodiment 1 Cloning of Fusarium asiaticum β-tubulin Gene

Synthesis of the Fusarium β-tubulin gene includes the following specific steps:

Fusarium-resistant β-tubulin gene was selected as a template to be artificially synthesized into β-tubulin gene by chemical synthesis, where the codon of the sequence encoding 198 amino acids was resistance loci of the benzimidazole agents, and the sequence is shown in SEQ ID NO.1 with the length of 2055 nt.

(DNA sequence of β-tubulin gene was obtained by isolation and corresponds to the sequence (accession number: FGSG_06611) published in NCBI gene database (https://www.ncbi.nlm.nih.gov/gene).

### Embodiment 2 Cloning of 2β-tubulin Gene

Synthesis of the Fusarium β-tubulin cDNA includes the following specific steps:

An intron sequence was removed according to the β-tubulin gene sequence in Embodiment 1, then β-tubulin gene cDNA was artificially synthesized by chemical synthesis, and the sequence (the DNA sequence obtained after removing intron) is shown in SEQ ID NO.2 with the length of 1686 nt.

### Embodiment 3 Construction of RNAi Vectors Specific to Different Segments of β-tubulin Gene cDNA and Fungal Transformation

(1) cDNA of the β-tubulin gene obtained from Embodiment 2 was divided into 4 different segments, respectively named βTub-1 (cDNA start-stop loci: 1 nt-482 nt), βTub-2 (cDNA start-stop loci: 460 nt-984 nt), βTub-3 (cDNA start-stop loci: 917 nt-1405 nt) and βTub-4 (cDNA start-stop loci: 1345 nt-1686 nt); Magnaporthe oryzae, start-stop loci of I3Tub-3cDNA were 922 nt-1409 nt, start-stop loci of βTub-4cDNA were 1345 nt-final base; Botrytis cinerea, start-stop loci of βTub-3cDNA were 727 nt-1209 nt, start-stop loci of βTub-4cDNA were 1150 nt-final base; Colletotrichum higginsianum, start-stop loci of βTub-3cDNA were 755 nt-1242 nt, start-stop loci of βTub-4cDNA were 1150 nt-final base; a specific PCR primer is designed to amplify a forward sequence of each segment, and each primer sequence is respectively as follows:

2Tub-1F:
(SEQ ID NO. 7)
GGGGACAAGTTTGTACAAAAAAGCAGGCTCTCCTTCATTTTACTTTTAG
GC
2Tub-1R:
(SEQ ID NO. 8)
GGGGACCACTTTGTACAAGAAAGCTGGGTAGCTCGGCACCCTCGGTGTA
AT
2Tub-2F:
(SEQ ID NO. 9)
GGGGACAAGTTTGTACAAAAAAGCAGGCTAACTGGGCCAAGGGTCATTA
CAC
2Tub-2R:
(SEQ ID NO. 10)
GGGGACCACTTTGTACAAGAAAGCTGGGTGACCAGTCAGAGGGGCAAAT
CC
2Tub-3F:
(SEQ ID NO. 11)
GGGGACAAGTTTGTACAAAAAAGCAGGCTAGCTCGCTGTTAACATGATT
CCG
2Tub-3R:
(SEQ ID NO. 12)
GGGGACCACTTTGTACAAGAAAGCTGGGTTGCAAGTTGGACTGGGCCTC
GG
2Tub-4F:
(SEQ ID NO. 13)
GGGGACAAGTTTGTACAAAAAAGCAGGCTGGCTTTCTTGCATTGGTACA
CAAG
2Tub-4R:
(SEQ ID NO. 14)
GGGGACCACTTTGTACAAGAAAGCTGGGTTTAGAAATCGTGCTTAAAAA
CAC

50 μL PCR reaction system: water was added to 20 ng of synthetic β-tubulin gene cDNA, 5 μL of 10×LA PCR buffer, 4 μL of 10 mM dNTP, 1 μL of forward and reverse primers respectively, 0.5 μL of LATaq enzyme to 50 μL (reagents for PCR reaction were purchased from TaKaRa (Da Lian)). PCR reaction conditions are: initial denaturation for 3 min at 95° C.; 95° C. 20 s, 56° C. 30 s, 72° C. 30 s for three cycles; extension for 10 min at 72° C.

PCR products were respectively mixed with a commercial donor vector pDONR201 after extracted by gel to form an entry vector containing a new site attL1/attL2, then the entry vector was mixed with a target vector pDestination respectively for specific recombination, and reaction products were transformed into Escherichia coli competence to form an interference expression vector of pβTubRNAi series: pβTubRNAi-1, pβTubRNAi-2, pβTubRNAi-3 and pβTubRNAi-4 (as shown in FIG. 1).

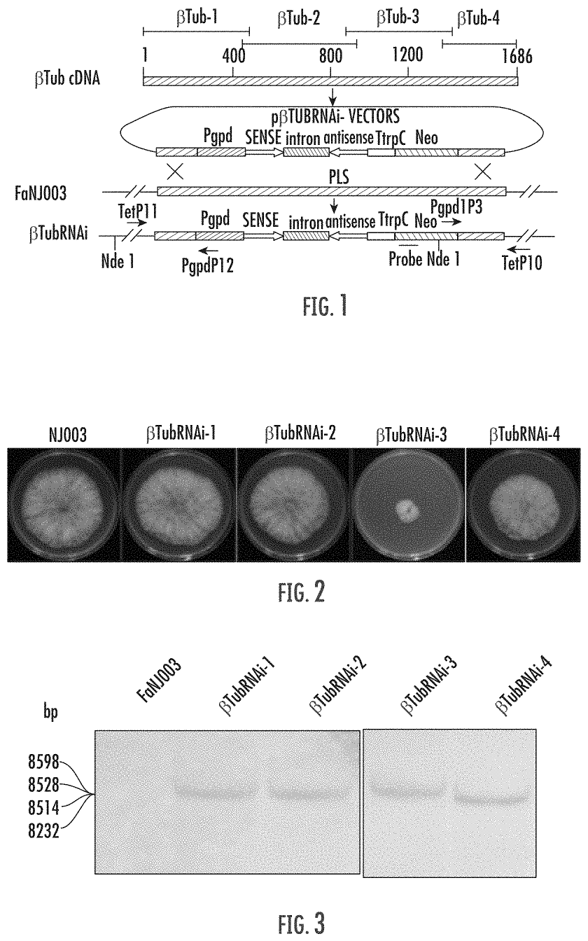

(2) 4 RNAi interference vectors obtained from step (1) were introduced into protoplast of benzimidazole-resistant strains NJ003 and sensitive strains 2021 to obtain 8 different recombinant strains by means of fungal protoplasts conversion (Maier etc., Development of a highly efficient gene targeting system for Fusarium graminearum using the disruption of apolyketide synthase gene as a visible marker. FEMS Yeast Res.2005.(5):653-662). Recombinant strains with NJ003 as an original strain were respectively named βTubRNAi-1, βTubRNAi-2, βTubRNAi-3 and βTubRNAi-4 strains, and recombinant strains with 2021 as an original strain were βTubRNAi-i, βTubRNAi-ii, βTubRNAi-iii and βTubRNAi-iv strains. Recombinant strains produced with 2021 as the original strain has consistent transformant phenotype with the one produced with NJ003 as the original strain, FIG. 2 shows phenotype of recombinant strains produced with NJ0031 as the original strain on a PDA medium.

### Embodiment 4 Screening of Effective RNAi Interference Segments of Fusarium graminearum β-tubulin Gene

### (1) Phenotype Experiment

A PDA medium plate was prepared, a 6 cm dish was poured onto a corresponding 7 ml medium, 10 μL 5×10 5/ml fresh conidia of the strains obtained from Embodiment 3 were taken and inoculated in the center of the medium plate, then the plate was put into an incubator at 25° C. for dark culture, colonial morphology was observed 3 d later, as shown in FIG. 2.

Results of FIG. 2 indicate that: βTubRNAi-3 and βTubRNAi-4 strains show decrease of biomass on the PDA medium, and biomass of the βTubRNAi-3 strain on the PDA medium decreases to the maximum extent. It indicates that the two segments of the β-tubulin gene may cause the decline of mycelial biomass and growth vigor after being interfered.

### (2) Southern Analysis

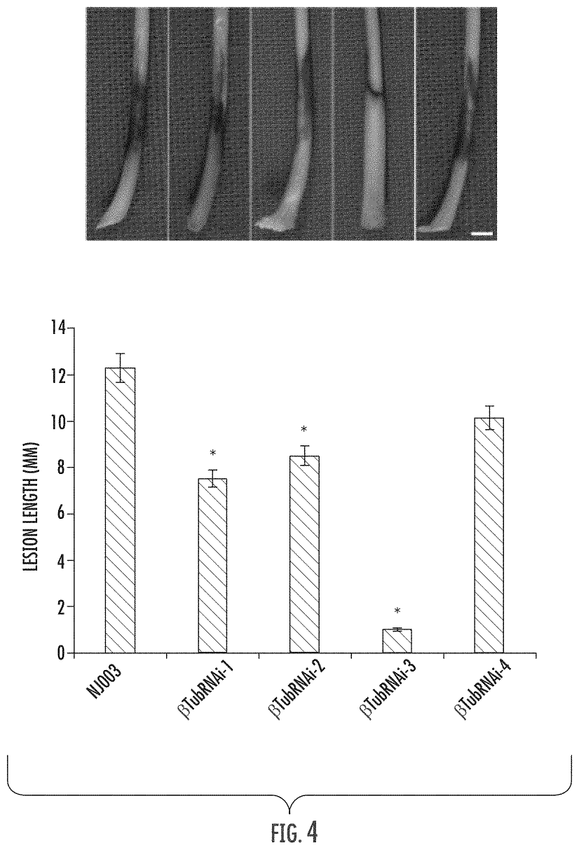

4βTubRNAi transformant strains obtained from Embodiment 3 were set as examples, genomes were respectively extracted by NcoI digestion and hybridized by a probe G418P-F/G418P-R, expected hybrid strips were respectively 8514 bp, 8598 bpm, 8528 bp, 8232 bp, and there was no strip in the NJ003 genome after being hybridized; transformants are single copies, and the size of the hybrid strips accords with the expected, showing that the vector has been correctly introduced into target gene, as shown in FIG. 3.

### (3) Experiment on Drug Sensitivity

8β-tubulin gene RNA-interference Fusarium transformants, benzimidazole-resistant strains NJ003 and sensitive strains 2021 obtained from Embodiment 3 were respectively set as analysis samples, the concentration of carbendazim in PDA medium was set 0, 0.1, 0.2, 0.4, 0.8, 1.6, 3.2, 6.4 and 12.8 μg/mL for measurement of resistant strains; the concentration of carbendazim in PDA medium was set 0, 0.01, 0.02, 0.04, 0.08, 0.16, 0.32, 0.64 and 1.28 μg/mL for measurement of sensitive strains, when the strains at the concentration of 0 μg/mL grew to 6 cm, EC50 values of the samples were measured by a PDA flat method.

Results of FIG. 1 indicate that: the RNA-interfered Fusarium transformants in different segments of the β-tubulin gene show significant reduction of EC50 values, indicating that after being interfered by RNA, different RNA segments of the β-tubulin gene may decrease the resistance of Fusarium to benzimidazole agents and enhance drug sensitivity (table 1).

TABLE 1
Analysis on the sensibility of β-tubulin gene RNAi transformants
to carbendazim

| | | EC50 |
|---|---|---|
| Strain and transformant | Correlation | (μg/ml) |
| NJ003 (β-tubulin binding agent-resistant strain | 0.9663 | 2.34 |
| βTubRNAi-1 (RNAi drug resistant strain) | 0.9736 | 1.69 |
| βTubRNAi-2 (RNAi drug resistant strain) | 0.9712 | 1.63 |
| βTubRNAi-3 (RNAi drug resistant strain) | 0.9384 | 0.7 |
| βTubRNAi-4 (RNAi drug resistant strain) | 0.9063 | 0.42 |
| 2021 (β-tubulin binding agent sensitive strain) | 0.9783 | 0.2 |
| βTubRNAi-i (RNAi sensitive strain) | 0.9632 | 0.12 |
| βTubRNAi-ii (RNAi sensitive strain) | 0.9745 | 0.11 |
| βTubRNAi-iii (RNAi sensitive strain) | 0.9458 | 0.07 |
| βTubRNAi-iv (RNAi sensitive strain) | 0.9268 | 0.04 |

### (4) Inoculation in Seedling Stage

Seeds of Yumai 35 wheat were taken, sterilized for 5 min by 0.1% mercury bichloride, washed by sterile water for 3 times and soaked for 2 h; seeds were placed into plastic boxes paved by double sterile filter paper with 25 seeds/box for dark culture for 3 d at 25° C. under the conditions of 90% humidity, 12 h illumination so that coleoptiles grew to 3 cm, then tips of the coleoptiles were fast cut by a sterilized scissors, 5 μL (5×105/mL, obtained from 4 recombinant strains with NJ003 as the original strain in Embodiment 3) spore liquor was respectively inoculated onto notches for culture in the same conditions, 7 d later, lesion length was surveyed and results were shown in table 2.

Pathogenicity analysis indicates that different segments of the β-tubulin gene may cause the decline of Fusarium pathogenicity after being interfered, showing remarkable decrease of lesion length.

FIG. 4 shows pictures of scab portions of 5 Yumai 35 seedlings. The pictures visually show that scab of the seedlings is significantly shorter than that of the seedlings in control group after RNA is interfered in different segments of the β-tubulin gene.

TABLE 2
Pathogenicity of β-tubulin RNAi strains inoculated into Yumai 35
seedlings in seedling stage

| Strain | Lesion length (cm) |
|---|---|
| NJ003 (Carbendazim-resistant strain) | 12.3 |
| βTubRNAi-1 | 7.5 |
| βTubRNAi-2 | 8.5 |
| βTubRNAi-3 | 1.0 |
| βTubRNAi-4 | 10.1 |

### (5) Measurement of Drug Sensitivity in Seedling Stage

Yumai 35 wheat served as a variety of field wheat for test, 50% carbendazim wettable powder was sprayed in flowering stage, the concentration was set 300 g ha-1, 600 g ha-1, 1200 g ha-1, after spraying for 24 h, highly benzimidazole agents-resistant NJ003 strains and 4β-tubulin gene RNA-interfered recombination strains were inoculated for 21 d for morbidity survey, and results were shown in table 3, represented by rate of infected spikelets:

Rate of infected spikelets=Number of infected spikelets/Total spikelets×100%

Difference analysis was performed to inoculation survey results by Student's tests and the multiple comparison of variance analysis.

The analysis indicates that after 300 g ha-1 50% carbendazim wettable powder is applied, the pathogenicity of the RNA-interfered strain decreases obviously relative to the high-resistant strain NJ003, and the sensitivity of the RNA-interfered strain to benzimidazole increases remarkably.

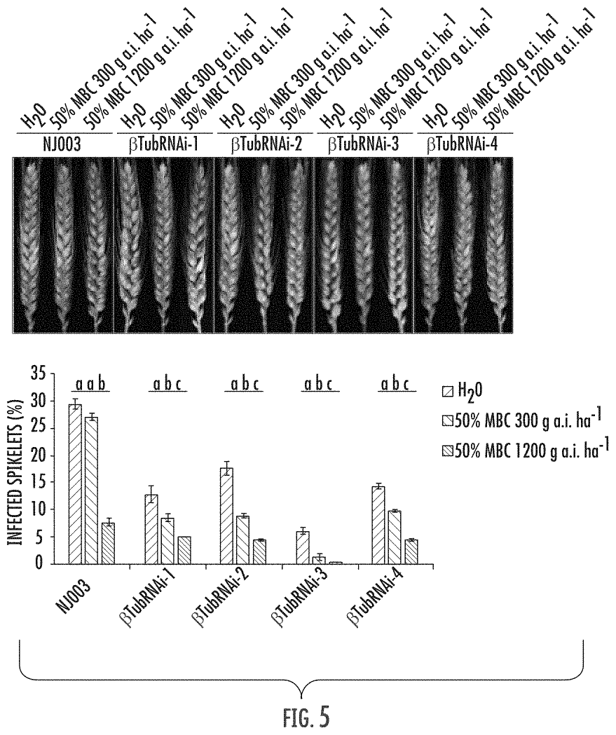

Results are shown in FIG. 5, when 300 g ha-1 50% carbendazim wettable powder is sprayed, the morbidity of resistant strains is not improved, while the pathogenicity of resistant strains decreases remarkably, showing significant decrease of sick ear; when 1200 g ha-1 50% carbendazim wettable powder is sprayed, the resistant strains have little pathogenicity, especially for recombinant fungi βTubRNAi-3, diseases only occur in the inoculated flowers instead of spreading to adjacent flowers. The analysis indicates that after 300 g ha-1 50% and 1200 g ha-1 carbendazim wettable powder are applied, the pathogenicity respectively decreases 26.7%-81.893%, 60%-100% obviously relative to the high-resistant strain NJ003, and the sensitivity of the strain to benzimidazole increases remarkably, as shown in table 3 and FIG. 5.

Table 3 shows measurement on field efficacy of a high-resistant strain NJ003 and a βTubRNAi transformant after being sprayed 50% wettable powder

| | Morbidity (%) after spraying 50% carbendazim | | |
|---|---|---|---|
| | wettable powder (g ha-1) | | |
| Strain | 0 | 300 | 1200 |
| NJ003 (Carbendazim- | 29.35 | 27.00 | 7.64 |
| resistant strain) | | | |
| βTubRNAi-1 | 12.76 | 8.44 | 4.92 |
| βTubRNAi-2 | 17.58 | 8.85 | 4.43 |
| βTubRNAi-3 | 6.03 | 1.16 | 0.28 |
| βTubRNAi-4 | 14.25 | 9.70 | 4.38 |

### Embodiment 5 Measurement of Field Efficacy of βTubdsRNA to Fusarium

### (1) Preparation of βTubdsRNA preparation

1) Full-length β-tubulin cDNA served as a template to synthesize 2 kinds of 850 bp dsRNAβTub-5(1 nt-850 nt) and 850 bp βTub-6 (836 nt-final base) of the β-tubulin gene, the dsRNA synthesis was conducted by a MEGAscript®RNAi Kit AM1626 kit from Invitrogen, sequences of the 2βTubdsRNA are the same as that of the corresponding template whose sequence behind T (thymine) was substituted by U (uracil);

2) Any of βTub-1, βTub-2, βTub-3 and βTub-4 or any combination of different segments served as a template of the synthesized segment of the in-vitro interference dsRNA to synthesize 14 kinds of βTubdsRNA, and the dsRNA synthesis was performed by a MEGAscript®RNAi Kit AM1626 kit from Invitrogen, the sequence of each of the 14 βTubdsRNA was the same as that of the corresponding template whose sequence behind T (thymine) is substituted by U (uracil);

3) Full-length β-tubulin cDNA served as a template to synthesize full-length βTubdsRNA by a MEGAscript®RNAi Kit AM1626 from Invitrogen, and SortCut®RNase III (from NEB, and steps were as shown in the specification of the kit) was used for digestion to produce 15-30 nt siRNA randomly.

The above 16 kinds of βTubdsRNA and a 15-30 nt siRNA were respectively diluted by an ammonium acetate buffer solution (ingredients: 0.1 M ammonium acetate, 0.75 mM EDTA, 4% ethylene glycol and 40 mM calcium chloride), and diluted to 2.5-320 ng/μl.

### (2) Isolation Interference Process of βTubdsRNA

The concentration of fresh Fusarium asiaticum spore was adjusted by a SNA medium (0.1% KH2PO4, 0.1% KNO3, 0.05% MgSO4.7H2O, 0.05% KCl, 0.02% glucose and 0.02% sucrose) and an ammonium acetate buffer solution (ingredients: 0.1 M ammonium acetate, 0.75 mM EDTA, 4% ethylene glycol and 40 mM calcium chloride) to 2000/mL. A mixed agent of 50 μL GFP-dsRNA (control), 16 kinds of 30 ngμL-1 βTubdsRNA and 1 15-30 nt siRNA or a mixed agent of 16 βTubdsRNA and 15-30 nt siRNA and 1 ngμL-1 benzimidazole carbendazim was added onto a 96-hole elisa plate, and the 1 ngμL-1 carbendazim carbendazim served as a control group to be mixed with 50 μL NJ003 spore liquor (2000/mL) well, cultured for 5 d at 25° C., then placed on an inverted microscope for observation.

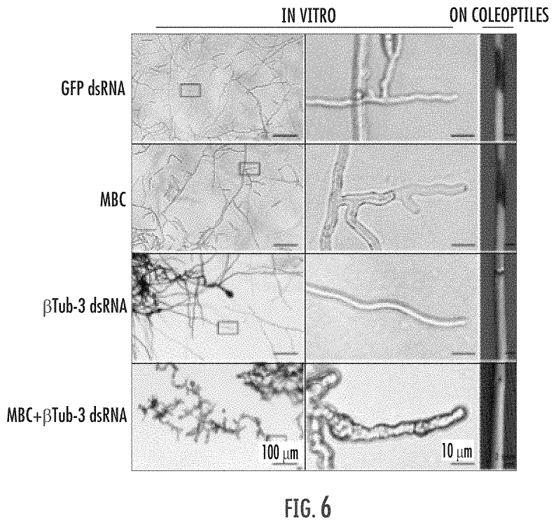

Results are shown in FIG. 6, in the combination of βTubdsRNA and 1 ngμL-1 carbendazim, mycelia grow slowly, show more obvious bending growth, have more branches and swelling structures compared with the process of applying carbendazim alone; after being treated by the combination of βTubdsRNA and 1 ngμL-1 carbendazim for 4 d, the mycelial still keep significant interference effect, while the mycelial treated by βTubdsRNA alone recover to normal morphology. It indicates that the benzimidazole may prolong drug retention period of dsRNA, and dsRNA may improve drug sensitivity to benzimidazole agents.

### (3) Measurement of βTubdsRNA Efficacy in Living Plants

Yumai 35 seeds were sterilized and soaked by sterile water for 2 h, then evenly paved into plastic boxes with double filter paper, illuminated for 12 h and cultured in the dark for 12 h at 25° C. and 90% humidity till coleoptiles grew to 3 cm, then tips of the coleoptiles were fast cut by a scissors, grouped and sampled, and sprayed by a mixed liquor of 16 βTubdsRNA and 1 15-30 nt siRNA or 16 βTubdsRNA and 1 15-30 nt siRNA with benzimidazole agent (1 ng/μL carbendazim was sprayed in control group), then naturally dried for 6 h, 5 μL (5×105/mL) Fusarium spore liquor was inoculated onto notches for culture in the same conditions, 4 d later, lesion length was surveyed.

Results are shown in FIG. 6, βTubdsRNA may effectively inhibit the invasion of Fusarium in coleoptiles, showing scarce coleoptile mycelial bending in growth; after treated by the combination of βTubdsRNA and benzimidazole agent, the coleoptile has lower pathogenicity and enhanced sensibility to benzimidazole agent.

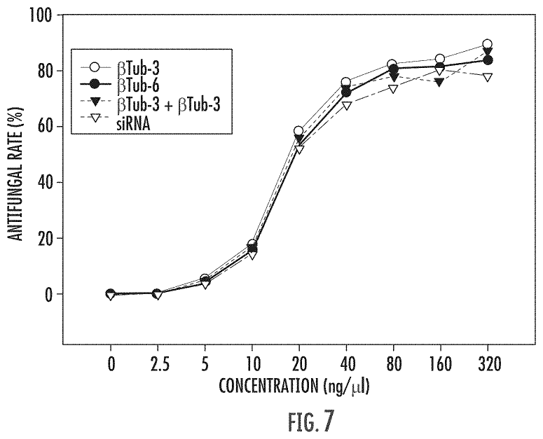

Results are shown in FIG. 6, 16 βTubdsRNA and 1 15-30 nt siRNA may effectively inhibit the morbidity of Fusarium in coleoptilea; compared with a single benzimidazole, the mixture of the 16 βTubdsRNA and 1 15-30 nt siRNA with benzimidazole agents may significantly reduce the lesion length of benzimidazole agents-resistant strains NJ003 on wheat coleoptiles, indicating that the 16 βTubdsRNA and 1 15-30 nt siRNA may enhance sensibility of Fusarium to benzimidazole agents on living plants of wheat. Each group shows good antifungal capacity against Fusarium within scope of siRNA concentration (2.5-320 ng/μl), where βTub-3, βTub-6 and siRNA were set as examples, a relation between RNA concentration and antifungal efficiency is shown in FIG. 7: βTubdsRNA or siRNA(5-320 ng/μl) at different concentrations may inhibit the morbidity of Fusarium, and have higher antifungal efficiency.

### Embodiment 6 Measurement of βTubdsRNA Antifungal Activity Against Different Fungi In Vitro

### (1) Synthesis of βTubdsRNA

The β-tubulin segment of β-tubulin gene is 15-30 nt siRNA obtained randomly by performing RNase digestion on a combination of segments βTubdsRNA and dsRNA or full-length or partial length of βTubdsRNA. BTubdsRNA synthesis was performed by a MEGAscript®RNAi Kit AM 1626 kit from Invitrogen (specific steps were shown in the specification of the kit), the sequence of βTubdsRNA is the same as that of the corresponding template whose sequence behind T (thymine) is substituted by U (uracil).

### (2) Interference Process in vitro

The concentration of Fusarium asiaticum, Fusarium graminearum, Fusarfum tricinctum, Fusarium oxysporum, Fusarium fujikuroi, Botrytis cinerea and Magnaporthe oryzae fresh spores was adjusted to 2000/mL by a SNA medium (0.1% KH2PO4, 0.1% KNO3, 0.05% MgSO4.7H2O, 0.05% KCl, 0.02% glucose and 0.02% sucrose) and a 0.1M ammonium acetate buffer solution, and the concentration of Colletotrichum higginsianum spores was adjusted to 2000/mL by a YEPD medium and 0.1 M ammonium acetate. GFP-dsRNA (control), 50 μL βTubdsRNA and 50 μL NJ003 spore liquor (2000/mL) were added to a 96-hole elisa plate, and mixedly well and cultured for 1 d and 4 d at 25° C., then placed on an inverted microscope for observation.

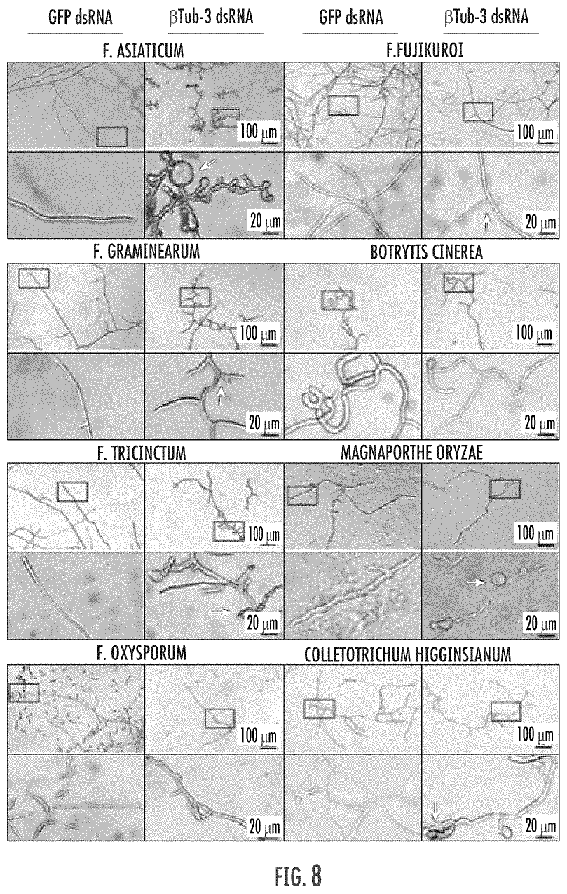

The results after cultured for 1 d are shown in FIG. 8, βTubdsRNA may significantly inhibit the growth or spore germination of various kinds of Fusarium, Fusarium fujikuroi, Botrytis cinerea, Magnaporthe oryzae and Colletotrichum higginsianum, causing delayed mycelial growth, increase of branches and occurrence of partial swelling structures.

### Embodiment 7 Experiment on βTubdsRNA Activity Against Fusarium, Magnaporthe oryzae, Botrytis cinerea and Colletotrichum higginsianum on Living Plants

The βTubdsRNA preparation (16 βTubdsRNA and 1 15-30 nt siRNA with the concentration of 40 ng/μL) in Embodiment 5 of the specification was respectively sprayed on the surface of rice leaves, cucumber leaves and soybean leaves, 30 ng/μL preparation was sprayed onto wheat coleoptiles, 0.1 M ammonium acetate was sprayed to the control group, 12 h later, 10 μL (1×105/mL) spore liquors of Magnaporthe oryzae, Botrytis cinerea, Colletotrichum higginsianum and 3 μL (1×106/mL) Fusarium spore liquor were inoculated respectively, each strain was inoculated on 40 leaves, and morbidity statistics was performed after 5 d, morbidity=number of sick leaves/number of gross inoculated leaves.

Experiment shows that the 16 βTubdsRNA and 1 15-30 nt siRNA may significantly improve the plant resistance to multiple pathogens, as shown in table 4.

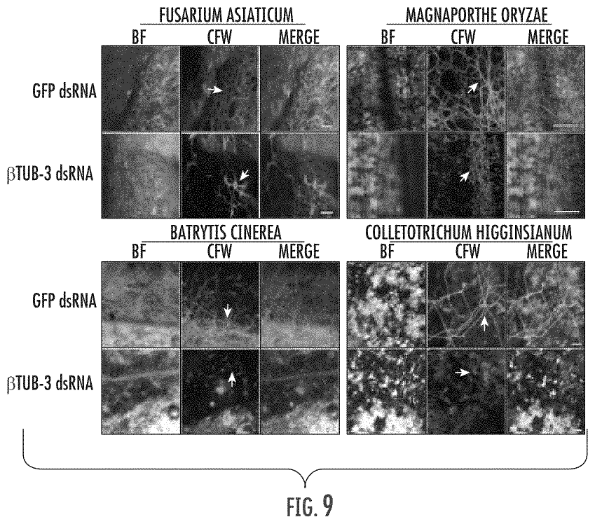

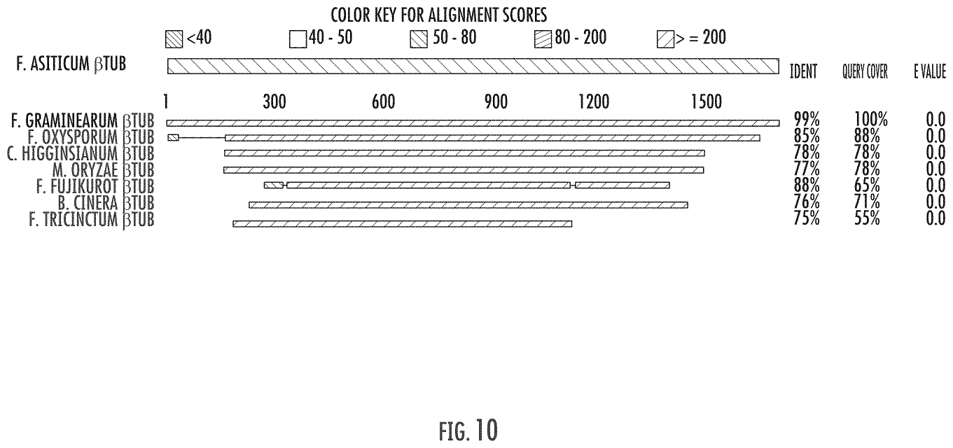

As shown in FIG. 9, βTubdsRNA was sprayed onto wheat coleoptiles to significantly inhibit the growth of Fusarium mycelium, showing few and scattered mycelia; after βTubdsRNA was sprayed onto wheat leaves, the formation of appressorium of Magnaporthe oryzae is obviously inhibited; and after βTubdsRNA was sprayed onto cucumber and soybean leaves, spore germination and mycelial growth of the soybean Colletotrichum higginsianum are inhibited obviously.

TABLE 4
Control efficiency of βTubdsRNA against Magnaporthe oryzae, Botrytis
cinerea and Colletotrichum higginsianum

| | Relative infection | |
|---|---|---|
| | volume of mycelia (%) | |
| | | βTub dsRNA |
| Inoculation mode of pathogens | Control | treatment |
| Wheat coleoptile | 100 | 8 |
| Magnaporthe oryzae inoculated on rice | 100 | 24 |
| leaves | | |
| Botrytis cinerea inoculated on cucumber | 100 | 20 |
| leaves | | |
| Colletotrichum higginsianum inoculated on | 100 | 44 |
| soybean leaves | | |

### Embodiment 8 Measurement of dsRNA Activity Derived from Different Fungi β-tubulin Gene Against Fusarium

(1) Homologous comparative analysis was performed to homologous genes of the Fusarium β-tubulin gene in different fungi, and the fungi include: F. asiaticum, F. graminearum, F. oxysporum, F. fujikuroi, F. tricinctum, F. oxysporum, Botrytis cinerea, Magnaporthe oryzae or Colletotrichum higginsianum. A NCBI online comparison tool BlastN was utilized for comparative analysis (https://blast.ncbi.nlm.nih.gov/Blast.cgi?PROGRAM=blastn&PAGE_TYPE=BlastSear ch&LINK_LOC=blasthome), analysis results were shown in FIG. 10, and the homology range was 75%-99%. PROGRAM=blastn&PAGE_TYPE=BlastSear ch&LINK_LOC=blasthome)

FIG. 10 shows homology of each fungus by different colors, where Colletotrichum higginsianum was 50-80, F. tricinctum and F. oxysporum were 80, and the rest fungi were >=200.

(2) β-tubulin cDNA of Magnaporthe oryzae, Botrytis cinerea and Colletotrichum higginsianum served as a template to synthesize dsRNA according to the process of the βTubdsRNA preparation in Embodiment 6, and the efficacy of dsRNA from different sources to Fusarium was measured by the process of measurement of βTubdsRNA efficacy on living plants, then Student's tests was utilized for difference analysis of the inoculation survey results. The results shown in table 5, indicates that βTubdsRNA from different sources has better antifungal activity and has no obvious difference compared with the Fusarium dsRNA by itself, denoting that the βTubdsRNA obtained from different fungal sources via a same method has equivalent efficacy among different fungi.

TABLE 5
Measurement of field efficacy of the βTubdsRNA from different
sources to Fusarium

| dsRNA source | Lesion length (cm) |
|---|---|
| Fusarium dsRNA | 0.61 ± 0.03 a |
| Magnaporthe oryzae dsRNA | 0.63 ± 0.03 a |
| Botrytis cinerea dsRNA | 0.67 ± 0.02 a |
| Colletotrichum higginsianum dsRNA | 0.62 ± 0.04 a |

### Embodiment 9 Disease Resistance Identification of βTubRNAi Transgenic Plants and an Application Thereof in Fungicide Reduction

### (1) Construction of βTubRNAi Expression Vectors in Plants

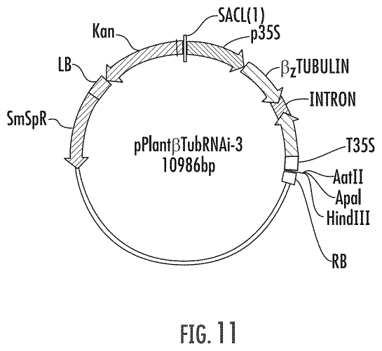

Commercial vectors pSGRNAi served as a framework, a cervical-loop structure composed of βTub-3 forward and reverse sequences βTub-3F: AGCTCGCTGTTAACATGATTCCG (SEQ ID NO. 11)); βTub-3R: TGCAAGTTGGACTGGGCCTCGG (SEQ ID NO. 12)) in Embodiment II and intron was linked to the downstream of 35S promoter and the upstream of 35S terminator to obtain a plant expression vector, named pPlantrβTub-RNAi-3, and the schematic diagram of the vector was shown in FIG. 11.

### (2) Genetic Transformation of Arabidopsis thaliana

A plant expression vector pPlantβTub-RNAi-3 was transformed to Arabidopsis thaliana (Colombia) by means of agrobacterium-mediated Arabidopsis thaliana transformation (refer to: Brian W. Tague and Joanna Mantis, In Planta Agrobacterium-Mediated Transformation by Vacuum Infiltration, 2006, 323:215-223), and resistant plants were obtained by screening kanamycin resistance.

### (3) Disease Resistance Identification of βTubRNAi Transgenic Arabidopsis thaliana and an Application Thereof in Fungicide Reduction

To detect whether the siRNA formed by βTubRNAi-3 transgenic Arabidopsis thaliana can inhibit the growth of invasive Fusarium and enhance drug susceptibility to benzimidazole, scab resistance of the transgenic plants was detected by leaves inoculation, the observation and statistics were conducted by phenotype of the inoculation, there was significant difference of the scab resistance between the non-transgenic control group and the transgenic strain (P<0.05). The scab area of the non-transgenic plants in the control group was 0.98 cm2, while scab area of the transgenic plants was 0.16 cm2 after Fusarium-resistant strain NJ003 was inoculated for 5 d, achieving 84% of control efficiency, morbidity of wild Arabidopsis thaliana plants after being sprayed 150 ng/μl carbendazim was 0.93 cm2, morbidity of wild Arabidopsis thaliana plants after being sprayed 800 ng/μl carbendazim was 0.51 cm2, while morbidity of wild Arabidopsis thaliana plants after being sprayed 10 ng/μl carbendazim was 0.01 cm2, achieving a 75-89% decline in morbidity, 99% control efficiency and significant decrease of fungicide dosage, as shown in table 6.

TABLE 6
Disease resistance identification of βTubRNAi transgenic Arabidopsis
thaliana and an application thereof in fungicide reduction:

| | Scab area of leave |
|---|---|
| Arabidopsis thaliana genotype/fungicide treatment | (cm2) |
| Wild Arabidopsis thaliana (Colombia) | 1.05 ± 0.03a |
| Wild Arabidopsis thaliana (Colombia)/sprayed | 1.00 ± 0.05a |
| 150 ng/μl carbendazim | |
| Wild Arabidopsis thaliana (Colombia)/sprayed | 0.54 ± 0.02b |
| 800 ng/μl carbendazim | |
| βTubRNAi-3 transgenetic Arabidopsis thaliana | 0.32 ± 0.11c |
| βTubRNAi-3 transgenetic Arabidopsis thaliana/ | 0.04 ± 0.00d |
| sprayed 150 ng/μl carbendazim | |

To sum up, the RNA interference technology of the β-tubulin gene of the present invention has green and safe advantages of increasing the drug sensitivity of pathogenic fungi, reducing the level of drug resistance, interfering with pathogenicity, enhancing plant disease resistance, controlling a variety of plant diseases, improving the drug sensitivity to a chemical agent (carbendazim), and prolonging the dsRNAs drug retention period by the carbendazim.

1. A β-tubulin segment of a β-tubulin gene, wherein the segment is 15-30 nt siRNA obtained randomly by performing RNase digestion on a combination of segments βTubdsRNA and dsRNA or full length or partial length of βTubdsRNA.

2. The β-tubulin segment of a β-tubulin gene according to claim 1, wherein the β-tubulin gene is from Fusarium asiaticum, Fusarium graminearum, Fusarium oxysporum, Fusarium fujikuroi, Fusarfum tricinctum, Botrytis cinerea, Magnaporthe oryzae and Colletotrichum higginsianum.

3. The β-tubulin segment of a β-tubulin gene according to claim 2, wherein the βTubdsRNA segment is obtained by dividing cDNA of the β-tubulin gene into 4 different segments, being respectively βTub-1 (cDNA start-stop loci: 1 nt-482 nt), βTub-2 (cDNA start-stop loci: 460 nt-984 nt), βTub-3 and βTub-4, wherein when the β-tubulin gene is from Fusarium graminearum or Fusarium asiaticum, cDNA start-stop loci of the βTub-3 are 917 nt-1405 nt, cDNA start-stop loci of the βTub-4 are 1345 nt-1686 nt; when the β-tubulin gene is from Magnaporthe oryzae, cDNA start-stop loci of the βTub-3 are 922 nt-1409 nt, and cDNA start-stop loci of the βTub-4 are 1345 nt-final base; when the β-tubulin gene is from Botrytis cinerea, cDNA start-stop loci of the (3Tub-3 are 727 nt-1209 nt, and cDNA start-stop loci of the βTub-4 are 1150 nt-final base; when the β-tubulin gene is from Colletotrichum higginsianum, and cDNA start-stop loci of the βTub-3 are 755 nt-1242 nt, cDNA start-stop loci of the βTub-3 are 1150 nt-final base.

4. The application according to claim 3, wherein corresponding dsRNA is synthesized in vitro with cDNA as a template, and a combination of the dsRNA segments is dsRNA obtained by an RNAi Kit with a combination of the multiple cDNA segments according to claim 3 as a template of a synthesized segment.

5. An application of the β-tubulin segment of a β-tubulin gene according to claim 1 in controlling plant diseases and/or enhancing plant disease resistance.

6. An application of the β-tubulin segment of a β-tubulin gene according to claim 1 in inhibiting the development of pathogens and pathogenicity.

7. An application of the β-tubulin segment of a β-tubulin gene according to claim 1 in enhancing drug sensitivity of pathogens to tubulin binding agents.

8. An in-vitro interference preparation, wherein the in-vitro interference preparation comprises the β-tubulin segment of a β-tubulin gene according to claim 5.

9. An application of the in-vitro interference preparation according to claim 8 in breeding transgenic plant resistant variety.

10. An application of the β-tubulin segment of a β-tubulin gene according to claim 2 in controlling plant diseases and/or enhancing plant disease resistance.

11. An application of the β-tubulin segment of a β-tubulin gene according to claim 3 in controlling plant diseases and/or enhancing plant disease resistance.

12. An application of the β-tubulin segment of a β-tubulin gene according to claim 4 in controlling plant diseases and/or enhancing plant disease resistance.

13. An application of the β-tubulin segment of a β-tubulin gene according to claim 2 in inhibiting the development of pathogens and pathogenicity.

14. An application of the β-tubulin segment of a β-tubulin gene according to claim 3 in inhibiting the development of pathogens and pathogenicity.

15. An application of the β-tubulin segment of a β-tubulin gene according to claim 4 in inhibiting the development of pathogens and pathogenicity.

16. An application of the β-tubulin segment of a β-tubulin gene according to claim 2 in enhancing drug sensitivity of pathogens to tubulin binding agents.

17. An application of the β-tubulin segment of a β-tubulin gene according to claim 3 in enhancing drug sensitivity of pathogens to tubulin binding agents.

18. An in-vitro interference preparation, wherein the in-vitro interference preparation comprises the β-tubulin segment of a β-tubulin gene according to claim 10.

19. An in-vitro interference preparation, wherein the in-vitro interference preparation comprises the β-tubulin segment of a β-tubulin gene according to claim 11.

20. An in-vitro interference preparation, wherein the in-vitro interference preparation comprises the β-tubulin -tubulin segment of a β-tubulin -tubulin gene according to claim 12.


SEQUENCE LISTING (18 sequences)

SEQ ID NO: 1 - DNA, 2055 bp, Fusarium asiaticum
ctccttcattttacttttaggcaccattttacgtgtgttttaaaggagacttttctttctcttgcctcgaccttcagtgaaacgaccagacccaaagacacctcacgtacaaacacaaacgcacacacacacacaacacattaatcaactaaatctgtcaagatgcgtgagattgtaagatttaacttctgactgacgctttgtcactcaaccaaactgactttttctttctcaggtccacgtccaggtcggccaatgtgtaagggcgaacccaccaccaaaaaaaaccttgagggacatatgttgagttgatgaatctttgtagggcaaccaagtcggttccagcttctggtaagtctttttttataagctttaagctcaattgaatccttgaaacctagatctaaccgtatttccaggtccaccgtctccaaggagcacggtattgatggcagcggcgcgtgagtcaacaacgtcaccgactttatgcctcacacttttgtctaacctgaacatcagataccacggaacttcagaccagcagcgtgagcgcatcaacgtctactttgctgaggtgagtaccaattgcagtatattgcaattgcaaatttcaacgtgattcctaactttctcagggcggcaacgacaagtacgtcccccgtgctgttctggtcgatcttgagtccggcccccaggatgccatccgcgccggccctctaggccagctgttccgccccgacaacttcgtcgccggtgaggccagtgccggaaacaactgggccaagggtcattacaccgagggtgccgagctcgttgaggaggccatcgatgttgttcgtcgcgaggttgagaactgtgaccaccttcagggtttccagctgacgcactctctcggcggtggtaccggttccggtatgggaacgcttcttctgtccaagatccgcgaggagttccccgatcgcatgatggccacctattccgttatgccctcgcccaaggtttccgacaccgttgttgaaccttacaacgccactttgtctctgaaccagctcgtcgagaactctgacctgacctactgtatcgataacgaggctctgtacgatatctacgagaggaccctcaagatcgccgatccttcgtacgccgatctcaactacctgatttccaccgtcatggccggtgtgacgacatgtttccgattccccggacagctcaactcggatctgcgaaagctcgctgttaacatgattccgttcccccgacttcacttcttcatggtcggatttgcccctctgactggtcgcaacatgaagactttccagcacgttaccgtccccggccttgctcagcagattttcgacaacaagaacatcatggccgctgccgatttccgcaacggacgatacctcgcttgttccgccatcttgtaagttttgaaactgaccaaatttcaaaaacacaaaaactaatgaaattcccagccgcggacgtctctcaacaaaggagatcgaggaccagatgctcaaggttcagaccaagaactccgagtactttgtcgactggatccccaacaacgtccaaacttccgtctgttccgtgcctccccgcggtctcgacatgtccgccactttcgttggcaactccaccgccgtccaggagatcttcaagcgtgtcgacgaccagttctcagccatgttccgacgcaaggctttcttgcattggtacacaagcgagggtatggacgagatggaattcaccgaggcccagtccaacttgcacgacttggtttccgagtaccagcaataccaagacgccgacatcgacgacgaggctgaggagtacgaggagggtgagcccgaggagtacgagggttgaatattggttgggaacgttggttaacatcatgactatttggtttatatcctcactactaccctcaaaatgtttctcatccccttcttcgttatacacacatttaaaacagaaattggttcaccaggcctgactaaattggccatattaataacaggcgtgtttttaagcacgatttctaaa

SEQ ID NO: 2 - DNA, 1686 bp, Fusarium asiaticum
ctccttcattttacttttaggcaccattttacgtgtgttttaaaggagacttttctttctcttgcctcgaccttcagtgaaacgaccagacccaaagacacctcacgtacaaacacaaacgcacacacacacacaacacattaatcaactaaatctgtcaagatgcgtgagattgtccacgtccaggtcggccaatgtggcaaccaagtcggttccagcttctggtccaccgtctccaaggagcacggtattgatggcagcggcgcataccacggaacttcagaccagcagcgtgagcgcatcaacgtctactttgctgagggcggcaacgacaagtacgtcccccgtgctgttctggtcgatcttgagtccggcccccaggatgccatccgcgccggccctctaggccagctgttccgccccgacaacttcgtcgccggtgaggccagtgccggaaacaactgggccaagggtcattacaccgagggtgccgagctcgttgaggaggccatcgatgttgttcgtcgcgaggttgagaactgtgaccaccttcagggtttccagctgacgcactctctcggcggtggtaccggttccggtatgggaacgcttcttctgtccaagatccgcgaggagttccccgatcgcatgatggccacctattccgttatgccctcgcccaaggtttccgacaccgttgttgaaccttacaacgccactttgtctctgaaccagctcgtcgagaactctgacctgacctactgtatcgataacgaggctctgtacgatatctacgagaggaccctcaagatcgccgatccttcgtacgccgatctcaactacctgatttccaccgtcatggccggtgtgacgacatgtttccgattccccggacagctcaactcggatctgcgaaagctcgctgttaacatgattccgttcccccgacttcacttcttcatggtcggatttgcccctctgactggtcgcaacatgaagactttccagcacgttaccgtccccggccttgctcagcagattttcgacaacaagaacatcatggccgctgccgatttccgcaacggacgatacctcgcttgttccgccatcttccgcggacgtctctcaacaaaggagatcgaggaccagatgctcaaggttcagaccaagaactccgagtactttgtcgactggatccccaacaacgtccaaacttccgtctgttccgtgcctccccgcggtctcgacatgtccgccactttcgttggcaactccaccgccgtccaggagatcttcaagcgtgtcgacgaccagttctcagccatgttccgacgcaaggctttcttgcattggtacacaagcgagggtatggacgagatggaattcaccgaggcccagtccaacttgcacgacttggtttccgagtaccagcaataccaagacgccgacatcgacgacgaggctgaggagtacgaggagggtgagcccgaggagtacgagggttgaatattggttgggaacgttggttaacatcatgactatttggtttatatcctcactactaccctcaaaatgtttctcatccccttcttcgttatacacacatttaaaacagaaattggttcaccaggcctgactaaattggccatattaataacaggcgtgtttttaagcacgatttctaaa

SEQ ID NO: 3 - DNA, 1686 bp, Fusarium graminearum
ctccttcattttacttttaggcaccattttacgtgtgttttaaaggagacttttctttctcttgcctcgaccttcagtgaaacgaccagacccaaagacacctcacgtacaaacacaaacgcacacacacacacaacacattaatcaactaaatctgtcaagatgcgtgagattgtccacgtccaggtcggccaatgtggcaaccaagtcggttccagcttctggtccaccgtctccaaggagcacggtattgatggcagcggcgcataccacggaacttcagaccagcagcgtgagcgcatcaacgtctactttgctgagggcggcaacgacaagtacgtcccccgtgctgttctggtcgatcttgagtccggcccccaggatgccatccgcgccggccctctaggccagctgttccgccccgacaacttcgtcgccggtgaggccagtgccggaaacaactgggccaagggtcattacaccgagggtgccgagctcgttgaggaggccatcgatgttgttcgtcgcgaggttgagaactgtgaccaccttcagggtttccagctgacgcactctctcggcggtggtaccggttccggtatgggaacgcttcttctgtccaagatccgcgaggagttccccgatcgcatgatggccaccttttccgttatgccctcgcccaaggtttccgacaccgttgttgaaccttacaacgccactttgtctctgaaccagctcgtcgagaactctgacgagaccttctgtatcgataacgaggctctgtacgatatctacgagaggaccctcaagatcgccgatccttcgtacgccgatctcaactacctgatttccaccgtcatggccggtgtgacgacatgtttccgattccccggacagctcaactcggatctgcgaaagctcgctgttaacatgattccgttcccccgacttcacttcttcatggtcggatttgcccctctgactggtcgcaacatgaagactttccagcacgttaccgtccccggccttgctcagcagattttcgacaacaagaacatcatggccgctgccgatttccgcaacggacgatacctcgcttgttccgccatcttccgcggacgtctctcaacaaaggagatcgaggaccagatgctcaaggttcagaccaagaactccgagtactttgtcgactggatccccaacaacgtccaaacttccgtctgttccgtgcctccccgcggtctcgacatgtccgccactttcgttggcaactccaccgccgtccaggagatcttcaagcgtgtcgacgaccagttctcagccatgttccgacgcaaggctttcttgcattggtacacaagcgagggtatggacgagatggaattcaccgaggcccagtccaacttgcacgacttggtttccgagtaccagcaataccaagacgccgacatcgacgacgaggctgaggagtacgaggagggtgagcccgaggagtacgagggttgaatattggttgggaacgttggttaacatcatgactatttggtttatatcctcactactaccctcaaaatgtttctcatccccttcttcgttatacacacatttaaaacagaaattggttcaccaggcctgactaaattggccatattaataacaggcgtgtttttaagcacgatttctaaa

SEQ ID NO: 4 - DNA, 1779 bp, Magnaporthe oryzae
atcggaccttttcctccctctcactaaaagaaaacccaccactgctaggaagcaacctctccttcacaaacacacaccacctttgcaagcatctctcaacttactctcgtactctctcgaccgcctcgctcccatcaacatcagaatccgacttgcaaaccataaaaatgcgtgaaattgttcaccttcagaccggccaatgcggcaaccaaattggtgctgctttctggcaaactatctctagcgagcacgggctcgacagcaatggagtttacaacggcacctccgagctccagctggagcgtatgagcgtctacttcaacgaggcctccggcaacaagcatgttccccgtgctgtcctcgtcgatctcgagcccggcaccatggacgccgtccgtgccggtccttttggccagctcttccgccccgacaacttcgtcttcggtcagtctggtgctggaaacaactgggccaagggtcactacactgagggtgccgagcttgtcgaccaggtccttgacgtcgtccgtcgtgaggctgagggctgtgactgcctccagggtttccagatcacccactccctgggtggtggtaccggtgccggtatgggtactctgctgatctccaagatccgcgaggagttccccgaccgtatgatggccaccttctcggtcgttccctcgcccaaggtttccgacaccgtcgttgagccctacaacgctaccctctcggtccaccagctggtcgagaactctgacgagaccttctgcattgacaacgaggctctgtacgacatctgcatgcgcaccctgaagctgtcgaacccctcatacggtgacctgaactacctggtttcggccgtcatgtctggcgtcaccacctgcttgcgtttccccggccagctcaactctgatctccgcaagcttgccgtcaacatggttcccttccctcgtctgcacttcttcatggttggcttcgctcctttgaccagccgtggtgcccactctttccgcgctgtcaccgttcccgagttgacccagcagatgttcgaccccaagaacatgatggctgcttctgacttcaggaatggtcgttacctgacctgctctgccatcttccgtggaaaggtttccatgaaggaggtcgaggaccagatgcgcaacgtccagaacaagaactcgtcgtacttcgtcgagtggatccccaacaacatccagaccgctctctgctctatcccgccccgcggcctcaagatgtcgtcgactttcatcggaaactcgaccgccatccaggagctgttcaagcgtgtcggtgagcagttcactgccatgttcaggcgcaaggctttcttgcattggtacactggtgagggtatggacgagatggagttcactgaggccgagtccaacatgaacgatcttgtttccgagtaccagcagtaccaggatgctggtgttgacgaggaggaagaggagtacgaggaggaggcccctcttgagggcgaggagtaggatcaaaacactcttaccccggatgcttcccaattggtcgcttacgatcaaaagtcggcacctttgtctccttgctccaggataacatcgctcgcggactcgctgtctgtgctcgagctggagggctgacgtagaggctgttgttttgattgtgctgacatgttggagactgttgtcgtaatttgatacctctcccagagacaatggagcaaaaagatacaatgttcattggacttgaatgcccttgccttagctatcgcgaggcccg

SEQ ID NO: 5 - DNA, 1281 bp, Botrytis cinerea
gacgtaggtacaattggtgctgctttctggcaaactatctctggcgagcacggtcttgacggttccggtgtctataatggtacatccgatctccaacttgagcgtatgaacgtctacttcaacgaggcttctggcaacaagtatgttccccgtgccgtcctcgtcgatttggagccaggtaccatggatgctgtccgtgccggtcctttcggtcaactcttccgtcccgacaacttcgttttcggtcaatctggtgctggtaacaactgggctaagggtcattacactgagggtgctgagcttgtcgaccaagttcttgatgttgtccgtcgtgaagctgaaggctgtgactgccttcaaggattccaaattacccactctctcggtggtggaactggtgccggtatgggtacgcttttgatctccaagatccgcgaggagttcccagatcgtatgatggctaccttctccgtcgtcccatcgccaaaggtttccgataccgttgtcgagccatataacgcaactctctctgtccatcaattggttgagaactctgacgagaccttctgtatcgataacgaggctctttacgatatttgcatgagaaccttgaagctcagcaacccatcttacggagatcttaaccacttggtttccgccgtcatgtccggtgttaccacctgtctccgtttccctggtcaacttaactcagatctccgaaagttggctgttaacatggttccattcccccgtctccatttcttcatggttggatttgctcctttgaccagtcgtggcgcacactctttccgtgctgtcaccgttccagagttgactcaacaaatgtacgaccctaagaacatgatggccgcttccgatttccgtaacggtcgttacttgacatgctctgccattttccgtggtaaggtttccatgaaggaggttgaggaccaaatgcgcaacgtccaaaacaagaactcatcctacttcgttgagtggatccctaacaacgtccaaaccgccctttgctccattcctccccgtggtctcaagatgtcctccaccttcgttggtaactcgacatccatccaagaacttttcaagcgtgtcggtgatcaattcactgctatgttcagaagaaaggctttcttgcattggtacactggtgaaggtatggacgagatggagttcactgaggctgagtccaacatgaacgatttggtttccgagtatcaacaataccaggatgcctcgatctctgaggagagagagtccataacg

SEQ ID NO: 6 - DNA, 1344 bp, Colletotrichum higginsianum
atgcgtgagatcgttcaccttcagaccggccagtgcggtaaccagattggtgctgccttttggcaaaacatctctggcgaacacggccttgacagcaatggcgtgtacaacggcacctctgagctccagctcgagcgcatgagcgtctacttcaacgaagcttccggcaacaagtatgtcccccgcgccgtcctcgtcgacttggagcccggtaccatggacgctgtccgcgctggccccttcggccagctcttccgccccgacaacttcgtcttcggccagtctggtgccggcaacaactgggccaagggtcactacaccgagggagctgagctcgtcgaccaggtcctcgacgtcgtccgccgcgaggctgagggctgtgactgcctccagggtttccagattacccactctctcggtggtggtaccggtgccggtatgggtaccctgctgatctccaagatccgcgaggagttccccgatcgcatgatggccaccttctccgtcgtcccctcccccaaggtctctgacactgttgttgagccttacaacgctactctttccgtccaccagctggtcgagaactcggacgagaccttctgcattgacaacgaggctctctacgacatctgcatgcgtactctcaagctttccaacccctcctacggcgacctgaaccaccttgtctctgccgtcatgtccggtgttaccacttgcctgcgtttccctggtcagctgaactctgacctgcgcaagctggccgtcaacatggttcctttcccccgtctccacttctttatggtcggcttcgctcccctgaccagccgtggtgcccactccttccgcgccgtcagcgttcccgagctcacccagcaaatgttcgaccccaagaacatgatggctgcttccgacttccgcaacggtcgctacctgacctgctctgccatcttccgtggtaaggtcgccatgaaggatgttgaggaccagatgcgcaacgtccagaacaagaactcttcctactttgtcgagtggattcccaacaacgtccagaccgctctctgctccatccccccccgcggtctcaagatgtcctccacttttgttggcaactctaccgccattcaggagctcttcaagcgtgtcggcgagcagttcaccgccatgttccgtcgcaaggctttcttgcattggtacactggtgagggtatggacgagatggagtttactgaggccgagtccaacatgaacgacttggtctccgagtaccagcagtaccaggacgctggtgttgacgaggaggaggaggagtacgaggaggatgtccctcttgaggaggaggtttaa

SEQ ID NO: 7 - DNA, 51 bp, artificial sequence
  2Tub-1F
ggggacaagtttgtacaaaaaagcaggctctccttcattttacttttaggc

SEQ ID NO: 8 - DNA, 51 bp, artificial sequence
  2Tub-1R
ggggaccactttgtacaagaaagctgggtagctcggcaccctcggtgtaat

SEQ ID NO: 9 - DNA, 52 bp, artificial sequence
  2Tub-2F
ggggacaagtttgtacaaaaaagcaggctaactgggccaagggtcattacac

SEQ ID NO: 10 - DNA, 51 bp, artificial sequence
  2Tub-2R
ggggaccactttgtacaagaaagctgggtgaccagtcagaggggcaaatcc

SEQ ID NO: 11 - DNA, 52 bp, artificial sequence
  2Tub-3F
ggggacaagtttgtacaaaaaagcaggctagctcgctgttaacatgattccg

SEQ ID NO: 12 - DNA, 51 bp, artificial sequence
  2Tub-3R
ggggaccactttgtacaagaaagctgggttgcaagttggactgggcctcgg

SEQ ID NO: 13 - DNA, 53 bp, artificial sequence
  2Tub-4F
ggggacaagtttgtacaaaaaagcaggctggctttcttgcattggtacacaag

SEQ ID NO: 14 - DNA, 52 bp, artificial sequence
  2Tub-4R
ggggaccactttgtacaagaaagctgggtttagaaatcgtgcttaaaaacac

SEQ ID NO: 15 - DNA, 45 bp, artificial sequence
  2Tub-5F
taatacgactcactatagggagactccttcattttacttttaggc

SEQ ID NO: 16 - DNA, 45 bp, artificial sequence
  2Tub-5R
taatacgactcactatagggagaaaatcaggtagttgagatcggc

SEQ ID NO: 17 - DNA, 45 bp, artificial sequence
  2Tub-6F
taatacgactcactatagggagaccaccgtcatggccggtgtgac

SEQ ID NO: 18 - DNA, 45 bp, artificial sequence
  2Tub-6R
taatacgactcactatagggagatttagaaatcgtgcttaaaaac